(19) United States
(12) Patent Application Publication
(10) Pub. No.: US 2019/0358238 A1
(43) Pub. Date: Nov. 28, 2019

(54) ALLOSTERIC ANTAGONISTS OF GPRC6a AND THEIR USE IN MITIGATING PROTEINOPATHIES

(71) Applicants: UNIVERSITY OF SOUTH FLORIDA, Tampa, FL (US); UNIVERSITY OF COPENHAGEN, COPENHAGEN (DK)

(72) Inventors: Daniel Carl Lee, Wesley Chapel, FL (US); Daniel Sejer Pedersen, Valby (DK); Hans Bräuner-Osborne, Copenhagen (DK); David Erik Gloriam, Hellerup (DK); Sebastiaan Kuhne, Amsterdam (NL); Henrik Karl Johansson, London (GB)

(21) Appl. No.: 16/461,716

(22) PCT Filed: Nov. 16, 2017
(86) PCT No.: PCT/US2017/062096

§ 371 Date: May 16, 2019

Related U.S. Application Data
(60) Provisional application No. 62/423,034, filed on Nov. 16, 2016.
(60) Provisional application No. 62/438,518, filed on Dec. 23, 2016.

(51) Int. Cl. A61K 31/5377

(52) U.S. Cl. CPC A61K 31/5377

(57) ABSTRACT

Disclosed herein are compounds and methods for antagonizing GPRC6a for the treatment of proteinopathies.

### CROSS-REFERENCE TO RELATED APPLICATION(S)

This application claims the benefit of and priority to U.S. Provisional Patent Application No. 62/423,034, filed on Nov. 16, 2016, and U.S. Provisional Patent Application No. 62/438,518, filed on Dec. 23, 2016, each of which is incorporated herein by reference in its entirety.

### FIELD

This disclosure relates to novel compounds and methods for antagonizing GPRC6a for the treatment of proteinopathies.

### INTRODUCTION

An emerging number of tauopathies including Alzheimer's disease (AD) continue to impact neuronal health and show causal impact on cognitive impairment and neuronal loss. The exact number of neurodegenerative diseases remains elusive, yet estimates project 600 brain disorders impacting 50 million Americans and costing in excess of $5 billion according to NIH. Currently, agents that modify disease or even slow progression fail to exist on the market for any of the tauopathies including AD. Strategies targeting disordered protein aggregates include increasing degradation (i.e., autophagy). Tauopathies include age-associated neurodegenerative diseases and remain a central target of AD, for which no disease-modifying treatments currently exist. Current therapies essentially provide symptomatic relief, yet disease progression continues to occur.

### SUMMARY

In an aspect, the disclosure relates to methods of treating a condition in a subject in need thereof. The methods may include administering to the subject a GPRC6a antagonist. In some embodiments, the condition is selected from proteinopathy, Alzheimer's disease, tauopathy, Parkinson's disease, synucleinopathy, prion disease, amyloidosis. TDP-43, and neurodegenerative disease. The GPRC6a antagonist as disclosed herein includes a compound of formula (I), or a pharmaceutically acceptable salt thereof,

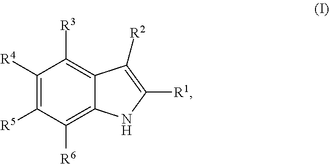

wherein

R¹ is hydrogen, alkyl, aryl, cycloalkyl, heteroaryl, or heterocycle, wherein the alkyl, aryl, cycloalkyl, heteroaryl, and heterocycle are each optionally substituted with one or more substituents selected from the group consisting of —OH, alkoxy, —NR^{1a}R^{1b}, halogen, nitro, —C(O)-alkyl, —C(O)—O-alkyl, —C(O)—NR^{1a}R^{1b};

R² is —X—(CR^{x}R^{y})ₘ₁—Y—(CR^{x}R^{y})ₘ₂—Z;

R³, R⁴, R⁵, R⁶ are independently hydrogen, alkyl, halogen, nitro, alkoxy, or alkyl substituted with —CO—R^{3a}, —CO—OR^{3a}, or —CO—NR^{3a}R^{3b},

wherein

X is —CH₂—, —CH(OH)—, or —CO—;

Y is —O— or —NR^{2a}—:

Z is hydrogen, -G, or —CO-G, wherein G is an optionally substituted aryl, optionally substituted cycloalkyl, optionally substituted heteroaryl, or optionally substituted heterocycle:

m1 is 0-10;

m2 is 0-10:

R^{1a}, R^{1b}, R^{3a}, and R^{3b} at each occurrence are independently hydrogen or alkyl;

R^{2a}, R^{x} and R^{y} at each occurrence are independently hydrogen or alkyl, or R^{2a} and one R^{x}, together with the N to which R^{2a} is attached and the C to which R^{x} is attached, form a 5-membered or 6-membered heterocycle.

In certain embodiments, disclosed is a method of treating condition selected from proteinopathy, Alzheimer's disease, tauopathy. Parkinson's disease, synucleinopathy, prion disease, amyloidosis, TDP-43, and neurodegenerative disease in a subject in need thereof, the method comprising administrating to the subject a compound of formula (I), or a pharmaceutically acceptable salt thereof.

In a further aspect, the disclosure relates to methods of inhibiting a GPRC6a in a subject. The methods may include administering to the subject a GPRC6a antagonist as detailed herein.

In some embodiments, the GPRC6a antagonist increases clearance of tau. In some embodiments, the GPRC6a antagonist reduces tau and/or alpha synuclein expression. In some embodiments, the GPRC6a antagonist reduces or clears multiple forms of tau and/or alpha synuclein. In some embodiments, the multiple forms are selected from insoluble, monomeric, and high molecular weight multimers. In some embodiments, the GPRC6a antagonist increases or promotes the clearance of pathogenic aggregation-prone proteins.

Another aspect of the disclosure provides a GPRC6a antagonist. Another aspect of the disclosure provides a GPRC6a allosteric antagonist.

The disclosure provides for other aspects and embodiments that will be apparent in light of the following detailed description and accompanying figures.

### BRIEF DESCRIPTION OF THE DRAWINGS

FIG. 1. L-arginine metabolic pathways including arginine decarboxylase (ADC), arginases (ARG), arginine, glycine amidotransferase (AGAT), nitric oxide synthases (NOS), and arginine deiminase (ADI). Arginine is essential for protein synthesis and amino acid turnover and may serve as a sensor for amino acid deprivation and autophagy activation through GPRC6a.

FIG. 2A, FIG. 2B, FIG. 2C, FIG. 2D, FIG. 2E, FIG. 2F, FIG. 2G, FIG. 2H, FIG. 2H, FIG. 2J, FIG. 2K, FIG. 2L, FIG. 2M, FIG. 2N, FIG. 2O, FIG. 2P, FIG. 2Q, FIG. 2R, FIG. 2S, FIG. 2T. Twelve-month-old rTg4510 tau transgenic mice or NonTg littermates received AAV9-GFP or AAV9-arginine deiminase (ADI) for two months. AAV9-ADI reduced hippocampal atrophy (FIG. 2A, FIG. 2B, FIG. 2C, FIG. 2G), p62 (FIG. 2D, FIG. 2E, FIG. 2F, FIG. 2H) suggesting increased autophagy, total tau (FIG. 2K, FIG. 2L. FIG. 2Q), and tangles (Gallyas silver) (FIG. 2M, FIG. 2N, FIG. 2R). There was no change in microglial staining of IBA-1 (FIG. 2O, FIG. 2P, FIG. 2S). Panels (FIG. 2I, FIG. 2J, FIG. 2T) show anti-hemagglutinin (HA) staining for a HA-fusion tagged ADI. (n=7-8) (p<0.05, student t-test or ANOVA).

FIG. 3A, FIG. 3B, FIG. 3C, FIG. 3D. C3H/htau cells treated with siRNA to GPRC6A show decreased tau expression compared to scrambled siRNA (FIG. 3A). GPRC6a allosteric antagonist Cpd#3 (Drug 47661) (0-250 tLM) decreases monomeric and high molecular weight (HMW) tau multimers (FIG. 3B) in C3H/htau cells. FIG. 3C-FIG. 3D show decreased tau in primary neurons after (Drug 47661) decreases tau at 10-100 nM. All treatments=72 hours (n=3 independent exp.).

FIG. 4. GPRC6a allosteric antagonist impacts tau levels and modifies autophagy markers in PS 19 tau transgenic mice. PSI9 (P301S) mice (bottom left of panel) show tau AT8 accumulation received an acute bolus injection of vehicle on one hemisphere and the antagonist (Drug 47661, 78 ng) on the opposite hemisphere for 72 hours. Western blot panel shows reduced tau expression (total tau) and several epitopes, decreased mTOR, p62 and increased beclin 1 suggesting induction of autophagy (n=3, each animal served as its own control to vehicle).

FIG. 5. Inducible tau shows photo conversion from green to red fluorescence. Panel shows stable inducible tetOn tau-Dendra2 expression over time following photoconversion from green to red fluorescence. Graph indicates real-time fluorescence after photoswtich (488 nm).

FIG. 6. C3H/htau cells treated with GPRC6A antagonist show decreased tau expression compared to vehicle (dotted line and first lane in each well). All GPRC6a allosteric antagonists Drug 47661 (red), PF020 (blue), PF037 (black) (0-100 μM) decreased monomeric and high molecular weight (HMW) tau multimers in C3H/htau cells at different concentrations. All treatments=72 hours (n=3 independent exp.).

FIG. 7. Show the overall procedure for SILAC based proteomics. Cells are grown and passaged in medium containing heavy and light amino acids using the Pierce SILAC Protein Quantitation Kit. Cells are treated with GPRC6a antagonist, vehicle. GPRC6a siRNA, or scramble siRNA. Lysates are mixed together, digested, fractioned, and analyzed by mass spectrometry. Data from the spectrometer will be processed using MaxQuant and Ingenuity Pathway Analysis (IPA).

FIG. 8A, FIG. 8B, FIG. 8C, FIG. 8D, FIG. 8E, FIG. 8F, FIG. 8G. Primary mouse cortico-hippocampal neurons were transfected with a LC3-mRFP-eGFP tandem plasmid construct for 48 hours (FIG. 8A-FIG. 8D). Primary neurons demonstrate a uniform difference in high versus low GFP and RFP fluorescence indicating different degrees of autophagy flux. The LC3-RFP/GFP tandem construct exploits differences between GFP (which is fluorescently quenched in lysosomes) and RFP (stable RFP fluorescence in lysosomes). Cells with more autophagosomes are labeled with yellow signal (i.e. RFP and GFP) and their maturation into autolysosomes labeled with a red/orange signal (i.e. RFP only, after quenching of GFP fluorescence in the lysosome), represents an indicator of autophagy activity. FIG. 8E-FIG. 8G represent mouse HT22 cells (immortalized hippocampal cell line) transfected with the LC3-RFP/GFP tandem construct and shows more heterogenic population, which exhibits various degrees of GFP/RFP expression (but overall yellow) in different compartments and organelles also indicating different degrees of autophagy flux.

FIG. 9. Graphs show relative half-life of Drug 47661. Naïve mice were injected with 5 mg/kg of Drug 47661 IV. Serial submandibular bleeds were taken 15 min and 60 min post IV injection. The half-life (0.16 h), volume of distribution (1.07 L/kg), and clearance rate (4.5 Lh/kg) were determined for Drug 47661. (N=3).

FIG. 10. Shown are BE(2) M17 cells stably transfected with wild-type alpha synuclein (16 kDa) treated with GPRC6a antagonist drug 47661 for 72 hours. Drug 47661 decreased monomeric (16 kDa) and oligomeric (>75 kDa) alpha synuclein expression, suggesting increased clearance or degradation of the protein relative to GAPDH as a protein loading control. Statistical analysis was performed using One-way Anova with Fisher's LSD multiple comparison test as post hoc. (n=3 independent experiments).

### DETAILED DESCRIPTION

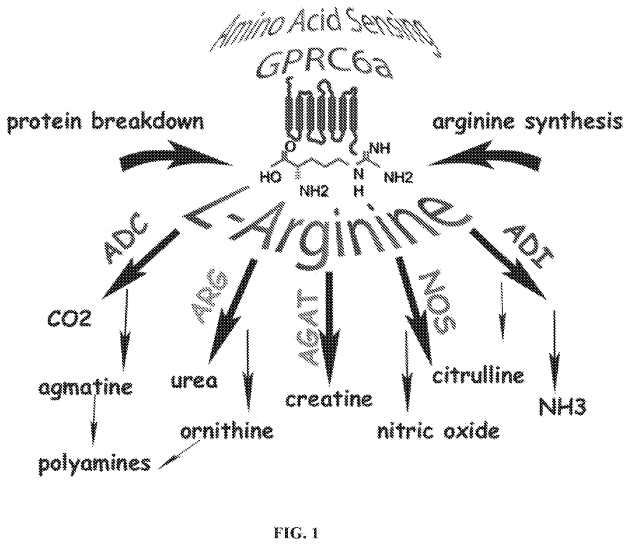

Detailed herein is the discovery of a unique interaction between arginine metabolism and tauopathies. Arginine metabolism is a branch-point affecting multiple biological processes and may have a considerable influence upon tau biology (FIG. 1). Several enzymes metabolize L-arginine including nitric oxide synthases (NOS), arginase 1 (Arg1), arginine decarboxylase (ADC), and arginine/glycine amidinotransferase (AGAT). Using cell and animal models of tauopathy, we have discovered the benefits of increasing Arg1 in reducing many aspects of the tau phenotype (J. B. Hunt, Jr. et al. J. Neurosci. 2015, 35, 14842). Arg1 overexpression significantly decreased the following components of the tau phenotype in vive: reduced phospho-tau by neurohistological measures, reduced tangle pathology, reduced atrophy, reduced phospho-tau species and nitrated tau by neurochemical measures, reduced high molecular weight tau/oligomers, reduced markers of inflammation, reduced inhibitors of autophagy, and reduced protein kinase activation. Since depletion of arginine may lead to increased autophagy through amino acid sensing, we mammalianized (codon usage) a bacterial enzyme arginine deiminase (ADI) to deplete L-arginine without making nitric oxide, polyamines, or agmatine and isolate the effects of L-arginine depletion. The mammalianized ADI reduced the tau phenotype. We then searched for receptors that modulate putative arginine signaling, and we examined GPRC6a.

Described herein is GPRC6a and its link to autophagy, amino acid sensing machinery, and the use of antagonists thereof to clear protein aggregates and treat proteinopathies. Also detailed herein is an inducible tetOn tau-Dendra2 photoswitchable cell line to measure degradation kinetics of tau. It was discovered that decreased signaling of GPRC6a increased tau and alpha synuclein clearance. With the discovery of a new class of compounds that modulate autophagy, the compositions and methods detailed herein may be used as new therapeutics for proteinopathies such as AD and other disorders of proteostasis. Further provided herein are GPRC6a antagonists that may be used to treat a condition such as a proteinopathy in a subject.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and.” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry. Thomas Sorrell, University Science Books, Sausalito, 1999; Smith and March March's Advanced Organic Chemistry, 5^{th} Edition, John Wiley & Sons, Inc., New York, 2001; Larock. Comprehensive Organic Transformations, VCH Publishers, Inc., New York, 1989; Carruthers, Some Modern Methods of Organic Synthesis, 3^{rd} Edition. Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 20 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means an unsaturated hydrocarbon chain containing from 2 to 20 carbon atoms and at least one carbon-carbon double bond.

The term “alkenyl” as used herein, means an unsaturated hydrocarbon chain containing from 2 to 20 carbon atoms and at least one carbon-carbon triple bond.

The term “alkoxyalkyl” as used herein, refers to an alkoxy group, as defined herein, appended to the parent molecular moiety through an alkylene group, as defined herein.

The term “arylalkyl” as used herein, refers to an aryl group, as defined herein, appended to the parent molecular moiety through an alkylene group, as defined herein.

The term “alkylene” as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—. —CH₂CH₂CH₂CH₂—, and —CH₂CH₂CH₂CH₂CH₂—.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl.

The term “carboxyl” as used herein, means a carboxylic acid, or —COOH.

The term “haloalkyl” as used herein, means an alkyl group, as defined herein, in which one, two, three, four, five, six, seven or eight hydrogen atoms are replaced by a halogen. Representative examples of haloalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “halogen” as used herein, means Cl, Br, I, or F.

The term “heteroalkyl,” as used herein, means an alkyl group, as defined herein. in which at least one of the carbons of the alkyl group is replaced with a heteroatom, such as oxygen, nitrogen, and sulfur.

The term “heteroaryl” as used herein, refers to an aromatic monocyclic ring or an aromatic bicyclic ring system. The aromatic monocyclic rings are five or six membered rings containing at least one heteroatom independently selected from the group consisting of N, O and S. The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyrazinyl, pyridinyl (including pyridin-2-yl, pyridin-3-yl, pyridin-4-yl), pyrimidinyl, thiazolyl, and quinolinyl.

The term “heterocycle” or “heterocyclic” as used herein means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N. and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihvdrobenzofuranyl, 2,3-dihydrobenzothienyl, 2,3-dihvdroisoquinoline, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but not limited to, octahydro-2,5-epoxypentalene, hexahydro-2H-2,5-methanocy clopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “heteroarylalkyl” as used herein, refers to a heteroaryl group, as defined herein, appended to the parent molecular moiety through an alkylene group, as defined herein.

The term “heterocycloalkyl” as used herein, refers to a heterocycle group, as defined herein, appended to the parent molecular moiety through an alkylene group, as defined herein.

The term “hydroxyl” or “hydroxyl” as used herein, means an —OH group.

In some instances, the number of carbon atoms in a hydrocarbyl substituent (e.g., alkyl or cycloalkyl) is indicated by the prefix “Cₓ-C_{y}-”, wherein x is the minimum and y is the maximum number of carbon atoms in the substituent. Thus, for example, “C₁-C₃-alkyl” refers to an alkyl substituent containing from 1 to 3 carbon atoms.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cvcloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

“Antagonist” refers to a compound that inhibits or reduces an activity of a polypeptide. An antagonist may indirectly or directly bind a polypeptide and inhibit the activity of the polypeptide, including binding activity or catalytic activity. For example, an antagonist may prevent expression of a polypeptide, or inhibit the ability of a polypeptide to mediate the binding of the polypeptide to a ligand. An “allosteric antagonist” refers to a compound that binds to a polypeptide at a secondary site, distinct from the primary ligand binding site, and inhibits or reduces an activity of the polypeptide.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifing a patient having CRC. A description of ROC analysis is provided in P. J. Heagerty, et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, or diseased after treatment, or a combination thereof.

The term “effective amount,” as used herein, refers to a dosage of the compounds or compositions effective for eliciting a desired effect. This term as used herein may also refer to an amount effective at bringing about a desired in vivo effect in an animal, preferably, a human, such as treatment of a disease.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quatemary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target.

“Subject” as used herein can mean a mammal that wants or is in need of the herein described therapies. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Target” as used herein can refer to an entity that a drug molecule binds. A target may include, for example, a small molecule, a protein, a polypeptide, a polynucleotide, a carbohydrate, or a combination thereof.

“Treatment” or “treating.” when referring to protection of a subject from a condition or a disease, means preventing, suppressing, repressing, ameliorating, or completely eliminating the condition or disease. Preventing the disease involves administering a composition of the present invention to a subject prior to onset of the condition or disease. Suppressing the condition or disease involves administering a composition of the present invention to a subject after induction of the disease but before its clinical appearance. Repressing or ameliorating the condition or disease involves administering a composition of the present invention to a subject after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide, to bind a ligand, or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 900%, 91%, 92%, 93%, 94%6, 95%, 96%, 97%, 98%, 99, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### 2. PROTEINOPATHIES

Proteinopathies are diseases or disorders in which a protein becomes structurally abnormal. For example, the protein may fail to properly fold into its normal configuration, e.g., become misfolded. Protein misfolding may include changes to the secondary and/or tertiary structure of a protein. For example, a protein may become structurally abnormal by increasing the beta-sheet secondary structure of the protein. The abnormal structure of the protein may disrupt its function, such as gaining a new function or losing normal function. The structurally abnormal protein may thereby disrupt the function of cells, tissues, and/or organs. Proteinopathies may also be referred to as proteopathies, protein confirmation disorders, or protein misfolding diseases. Proteinopathies include, for example, tauopathies and synucleopathies. Proteinopathies may also include prion disease and amyloidosis.

Tauopathies are neurodegenerative diseases associated with the aggregation of tau protein. Tau may be found in neurons of the central nervous system. In its native form, tau is a protein that is associated with microtubules and interacts with tubulin to stabilize microtubules and promote tubulin assembly into microtubules. In a tauopathy, the tau protein may be aggregated into neurofibrillary or gliofibrillary tangles in the brain and no longer stabilizes microtubules properly. The tangles may be formed by hyperphosphorylation of tau, which may cause tau to form insoluble aggregates. In some embodiments, the multiple forms of tau are selected from insoluble, monomeric, and high molecular weight multimers. Tauopathies include, for example, primary age-related tauopathy (PART)/Neurofibrillary tangle-predominant senile dementia, chronic traumatic encephalopathy including dementia pugilistica, progressive supranuclear palsy, Pick's Disease, corticobasal degeneration, some forms of frontotemporal lobar degeneration, frontotemporal dementia and parkinsonism linked to chromosome 17, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, Huntington's Disease, and Alzheimer's Disease (AD).

Synucleinopathies are neurodegenerative diseases characterized by the abnormal accumulation of aggregates of alpha-synuclein in, for example, neurons, nerve fibres, or glial cells. Alpha-synuclein may be found in the heart, muscle, brain, and other tissues. In the brain, alpha-synuclein may be found at the tips of neurons at the presynaptic terminal. Alpha-synuclein is a protein that can interact with phospholipids and proteins. Alpha-synuclein can directly bind to lipid membranes, by associating with the negatively charged surfaces of phospholipids. Alpha-synuclein may play a role in maintaining a supply of synaptic vesicles in presynaptic terminals by clustering synaptic vesicles. Alpha-synuclein may help regulate the release of dopamine. Alpha-synuclein may interact with tubulin and have activity as a microtubule-associated protein, similar to tau. In some embodiments, the multiple forms of alpha-synuclein are selected from insoluble, monomeric, and high molecular weight multimers. Synucleinopathies include, for example, Parkinson's Disease, dementia with Lewy bodies, neuroaxonal dystrophies, and multiple system atrophy. In some embodiments, synucleinopathies may overlap with tauopathies, potentially because of an interaction between alpha-synuclein and tau.

As detailed in the Examples, the GPRC6a receptor was identified as a drug target for proteinopathies, which may be used to treat proteinopathies such as certain neurodegenerative diseases that harbor protein aggregation and ultimately cell demise.

### 3. GPRC6A

G-protein-coupled receptors (GPCRs) are a large protein family of receptors that sense molecules outside the cell and activate inside signal transduction pathways and cellular responses. GPCRs are also known as seven-transmembrane domain receptors, 7TM receptors, heptahelical receptors, serpentine receptor, and G protein-linked receptors (GPLR) Coupling with G proteins. GPCRs are called seven-transmembrane receptors because they are integral membrane proteins that pass through the cell membrane seven times. GPCRs bind ligands that may include, but are not limited to, small molecules, proteins, peptides, polypeptides, nucleotides, polynucleotides, carbohydrates, lipids, and combinations thereof.

G-protein-coupled receptor family C group 6 member A (GPRC6a) is a protein that in humans is encoded by the GPRC6A gene. GPRC6a is a polypeptide that functions as a receptor of L-alpha-amino acids, cations (such as calcium), osteocalcin, and steroids. In some embodiments, GPRC6a binds L-α amino acids, particularly basic amino acids including L-arginine (high affinity), omithine (high affinity), and L-lysine. GPRC6a is also a membrane androgen receptor.

In some embodiments, GPRC6a governs extracellular amino acid abundance. GPRC6a may remain tonically activated and sense extracellular amino acid abundance of L-α amino acids but may become more sensitive to L-arginine and omithine during neurodegenerative conditions. In some embodiments, GPRC6a regulates energy metabolism. In some embodiments, GPRC6a governs protein turnover and clearance of unwanted protein aggregates, for example, in the context of neurodegenerative diseases. As detailed in the Examples, a novel allosteric antagonist to GPRC6a significantly increased the clearance of monomeric, insoluble, and oligomeric tau in stably overexpressing cells. Accordingly, in some embodiments, inhibition or antagonism of GPRC6a may increase or activate autophagy, tau clearance, and/or alpha synuclein clearance.

a. GPRC6a Antagonist

Further provided herein are antagonists of GPRC6a. In some embodiments, the GPRC6a antagonist is an allosteric antagonist. The GPRC6a antagonist as disclosed herein may increase or promote the clearance of pathogenic aggregation-prone proteins. The GPRC6a antagonist may reduce or clear multiple forms of tau and/or alpha synuclein. The GPRC6a antagonists may improve behavioral and pathological outcomes associated with tauopathies and synucleinopathy phenotypes.

The GPRC6a antagonist suitable for the compositions and methods as disclosed herein may include compounds that are known to have certain GPRC6a antagonist activities. Suitable GPRC6a antagonists may include those described in Johansson et al., Selective Allosteric Antagonists for the G Protein-Coupled Receptor GPRC6A Based on the 2-Phenylindole Privileged Structure Scaffold, J. Med. Chem. 2015, 58, 8938-8951.

In certain embodiments, the GPRC6a antagonist as disclosed herein is a compound of formula (I), or a pharmaceutically acceptable salt thereof,

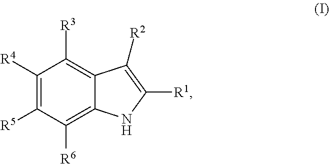

wherein

R¹ is hydrogen, alkyl, aryl, cycloalkyl, heteroaryl, or heterocycle, wherein the alkyl, aryl, cycloalkyl, heteroaryl, and heterocycle are each optionally substituted with one or more substituents selected from the group consisting of —OH, alkoxy, —NR^{1a}R^{1b}, halogen, nitro. —C(O)-alkyl, —C(O)—O-alkyl, —C(O)—NR^{1a}R^{1b};

R² is —X—(CR^{x}R^{y})ₘ₁—Y—(CR^{x}R^{y})ₘ₂—Z:

R³, R⁴, R⁵, R⁶ are independently hydrogen, alkyl, halogen, nitro, alkoxy, or alkyl substituted with —CO—R^{3a}, —CO—OR^{3a}, or —CO—NR^{3a}R^{3b},

wherein

X is —CH₂—, —CH(OH)—, or —CO—,

Y is —O— or —NR^{2a}—;

Z is hydrogen, -G, or —CO-G, wherein G is an optionally substituted aryl, optionally substituted cycloalkyl, optionally substituted heteroaryl, or optionally substituted heterocycle;

m1 is 0-10;

m2 is 0-10;

R^{1a}, R^{1b}, R^{3a}, and R^{3b} at each occurrence are independently hydrogen or alkyl;

R^{2a}, R^{x} and R^{y} at each occurrence are independently hydrogen or alkyl, or R^{2a} and one R^{x}, together with the N to which R^{2a} is attached and the C to which R^{x} is attached, form a 5-membered or 6-membered heterocycle.

In certain embodiments, R¹ is an optionally substituted aryl, optionally substituted cycloalkyl, optionally substituted heteroaryl, or optionally substituted heterocycle. In certain embodiments, R¹ is an optionally substituted aryl, such an unsubstituted or optionally substituted phenyl. In certain embodiments, R¹ is an unsubstituted phenyl. In other embodiments, R¹ is a phenyl substituted with one or more alkoxy or halogen.

In certain embodiments, X is —CO—.

In certain embodiments, Y is —NR^{2a}—.

In certain embodiments, m1 is 0, 1, 2, 3, or 4. In certain embodiments, m1 is 1 or 2.

In certain embodiments, m2 is 0, 1, 2, 3, or 4. In certain embodiments, m2 is 1 or 2.

In some embodiments, Z is -G or —CO-G.

In some embodiments, G is an optionally substituted heterocycle. In some embodiments, G is an optionally substituted heterocycle, which contains one or more N atoms. In some embodiments, G is an optionally substituted heterocycle, which contains one or more N atoms and is attached to the parent molecule through one N atom. In some embodiments, G is

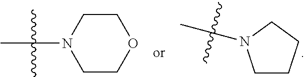 [SMILES: *N1CCOCC1 ; *N1CCCC1]

In certain embodiments, G is

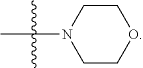 [SMILES: *N1CCOCC1]

In certain embodiments, Z is

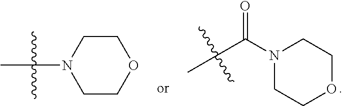 [SMILES: *N1CCOCC1 ; *C(=O)N1CCOCC1]

In some embodiments, R³, R⁴, R⁵, and R⁶ are independently hydrogen, alkyl, halogen, alkoxy, or alkyl substituted with —CO—NR^{3a}R^{3b}. In some embodiments, R³, R⁴, R⁵, and R⁶ are independently hydrogen, halogen, or alkoxy. In some embodiments, R³, R⁴, R⁵, and R⁶ are hydrogen. In some embodiments, R³. R⁵, and R⁶ are hydrogen, and R⁴ is halogen or alkoxy.

In some embodiments, R^{1a}, R^{1b}, R^{3a}, and R^{3b} are hydrogen, In some embodiments, R^{1a}, R^{1b}, R^{3a}, and R^{3b} at each occurrence are independently hydrogen or alkyl, such as C₁-C₄ alkyl.

In certain embodiments, R^{2a} is alkyl, such as C₁-C₄ alkyl. In some embodiments, R^{2a} is methyl.

In certain embodiments, R^{x} and R^{y} are hydrogen. In some embodiments, R^{x} and R^{y} at each occurrence are independently hydrogen or alkyl, such as C₁-C₄ alkyl.

In certain embodiments, R^{2a} and one R^{x}, together with the N to which R^{2a} is attached and the C to which R^{x} is attached, form a 5-membered or 6-membered heterocycle. In certain embodiments, the heterocycle formed by R^{2a} and one R^{x}, together with the N to which R^{2a} is attached and the C to which R^{x} is attached, is

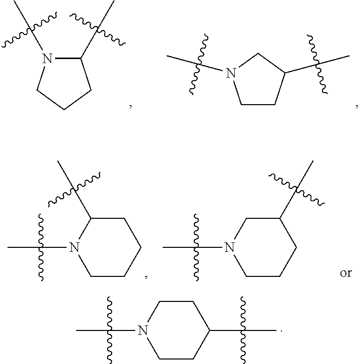

The heterocycle formed by R^{2a} and one R^{x}, together with the N to which R^{2a} is attached and the C to which R^{x} is attached may contain one or more heteroatoms in addition to the N to which R^{2a} is attached. Such heterocycle may be optionally substituted with one or more substituent groups disclosed herein, such as alkyl, alkoxy, or halogen.

In some embodiments, the GPRC6a antagonist is a compound of formula (I), wherein formula (I) is formula (I-a), or a pharmaceutically acceptable salt thereof.

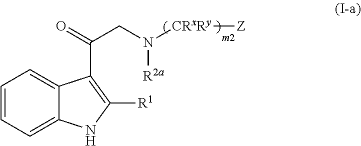

wherein

R¹ is optionally substituted aryl, optionally substituted cycloalkyl, optionally substituted heteroaryl, or optionally substituted heterocycle;

Z is -G or —CO-G;

R^{2a}, R^{x}, R^{y}, m2, and G are as defined above.

In some embodiments, the GPRC6a antagonist is a compound of formula (I-a), wherein R¹ is an optionally substituted aryl, such an unsubstituted or substituted phenyl. For example, in certain compounds of formula (I-a), R¹ is an unsubstituted phenyl, or R¹ is a phenyl substituted with one or more alkoxy or halogen.

In some embodiments, the GPRC6a antagonist is a compound of formula (I-a), wherein R^{2a} is alkyl, or R^{2a} and one R^{x}, together with the N to which R^{2a} is attached and the C to which R^{x} is attached, form a 5-membered or 6-membered heterocycle. In some embodiments, the GPRC6a antagonist is a compound of formula (I-a), wherein R^{2a} is alkyl. In some embodiments, the GPRC6a antagonist is a compound of formula (I-a), wherein R^{2a} and one R^{x}, together with the N to which R^{2a} is attached and the C to which R^{x} is attached, form a 5-membered or 6-membered heterocycle.

In some embodiments, the GPRC6a antagonist is a compound of formula (I-a), wherein R¹ is phenyl or phenyl substituted with one or more alkoxy or halogen, and R^{2a} is alkyl, or R^{2a} and one R^{x}, together with the N to which R^{2a} is attached and the C to which R^{x} is attached, form a 5-membered or 6-membered heterocycle.

In some embodiments, the GPRC6a antagonist is a compound of formula (I-a), wherein R¹ is phenyl, and R^{2a} is alkyl.

In some embodiments, the GPRC6a antagonist is a compound of formula (I-a), wherein R¹ is phenyl, and R^{2a} and one R^{x}, together with the N to which R^{2a} is attached and the C to which R^{x} is attached, form a 5-membered or 6-membered heterocycle.

In some embodiments, the GPRC6a antagonist is a compound of formula (I-a), wherein R¹ is phenyl substituted with one or more alkoxy or halogen, and R^{2a} is alkyl.

In some embodiments, the GPRC6a antagonist is a compound of formula (I-a), wherein m2 is 1, 2, 3, or 4. In some embodiments, the GPRC6a antagonist is a compound of formula (I-a), wherein m2 is 2.

In some embodiments, the GPRC6a antagonist is a compound of formula (I-a), wherein Z is -G or —CO-G, and G is an optionally substituted heterocycle. In some embodiments, the GPRC6a antagonist is a compound of formula (I-a), wherein Z is

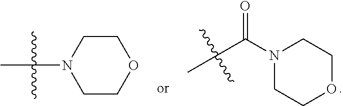 [SMILES: *N1CCOCC1 ; *C(=O)N1CCOCC1]

In some embodiments, the GPRC6a antagonist is a compound of formula (I-a), wherein formula (I-a) is formula (I-a1), (I-a2), or (I-a3), or a pharmaceutically acceptable salt thereof,

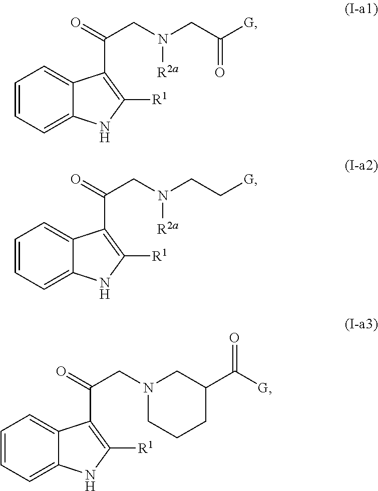

wherein R¹, R^{2a}, and G are as defined above.

In some embodiments, the GPRC6a antagonist is a compound of formula (I-a1), (I-a2), or (I-a3), wherein R¹ is optionally substituted aryl, such an unsubstituted or substituted phenyl. In some embodiments, the GPRC6a antagonist is a compound of formula (I-a1), (I-a2), or (1-a3), wherein R¹ is phenyl or phenyl substituted with one or more alkoxy or halogen.

In some embodiments, the GPRC6a antagonist is a compound of formula (I-a1), (I-a2), or (I-a3), wherein G is

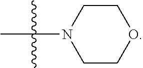 [SMILES: *N1CCOCC1]

In some embodiments, the GPRC6a antagonist is a compound of formula (I-b), or a pharmaceutically acceptable salt thereof,

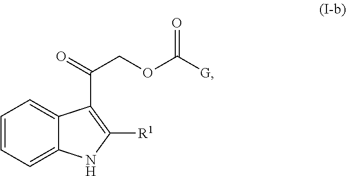

wherein R¹ and G are as defined above.

In some embodiments, the GPRC6a antagonist is a compound of formula (I-b), wherein R¹ is an optionally substituted aryl, such an unsubstituted or substituted phenyl. For example, in certain compounds of formula (I-b), R¹ is an unsubstituted phenyl, or R¹ is a phenyl substituted with one or more alkoxy or halogen.

In some embodiments, the GPRC6a antagonist is a compound of formula (I-b), wherein G is an optionally substituted aryl or optionally substituted heteroaryl. In some embodiments, the GPRC6a antagonist is a compound of formula (I-b), wherein G is an optionally substituted phenyl or pyrazinyl.

Representative compounds of formula (I) include, but are not limited to, the following compounds, or a pharmaceutically acceptable salt thereof shown in TABLE 1:

TABLE 1
Representative compounds of formula (I).

| Name | Structure |
|---|---|
| 2-(methyl(2-morpholino-2-oxoethyl)amino)-1- (2-phenyl-1H-indol-3-yl)ethan-1-one | 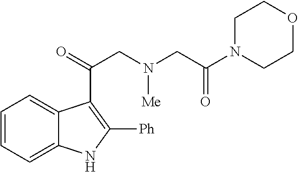 [SMILES: CN(CC(=O)c1c(-c2ccccc2)[nH]c2ccccc12)CC(=O)N1CCOCC1] |
| 1-(2-(4-methoxyphenyl)-1H-indol-3-yl)-2- (methyl(2-morpholino-2-oxoethyl)amino)ethan- 1-one | 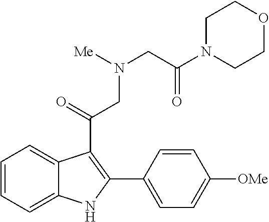 [SMILES: COc1ccc(-c2[nH]c3ccccc3c2C(=O)CN(C)CC(=O)N2CCOCC2)cc1] |
| 2-(methyl(2-morpholinoethyl)amino)-1-(2- phenyl-1H-indol-3-yl)ethan-1-one | 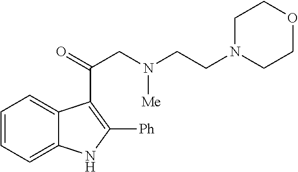 [SMILES: CN(CCN1CCOCC1)CC(=O)c1c(-c2ccccc2)[nH]c2ccccc12] |
| 2-(3-(morpholine-4-carbonyl)piperidin-1-yl)-1- (2-phenyl-1H-indol-3-yl)ethan-1-one | 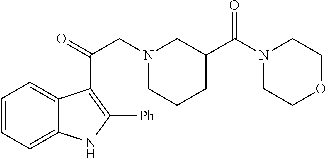 [SMILES: O=C(CN1CCCC(C(=O)N2CCOCC2)C1)c1c(-c2ccccc2)[nH]c2ccccc12] |
| 1-(2-(4-fluorophenyl)-1H-indol-3-yl)-2- (methyl(2-morpholino-2-oxoethyl)amino)ethan- 1-one | 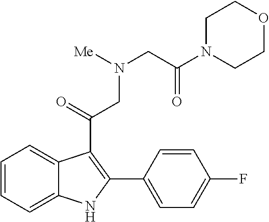 [SMILES: CN(CC(=O)c1c(-c2ccc(F)cc2)[nH]c2ccccc12)CC(=O)N1CCOCC1] |
| 1-(5-fluoro-2-phenyl-1H-indol-3-yl)-2- (methyl(2-morpholino-2-oxoethyl)amino)ethan- 1-one | 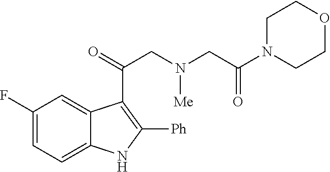 [SMILES: CN(CC(=O)c1c(-c2ccccc2)[nH]c2ccc(F)cc12)CC(=O)N1CCOCC1] |
| 2-((2-(1H-indol-3-yl)-2- oxoethyl)(methyl)amino)-1-morpholinoethan-1- one | 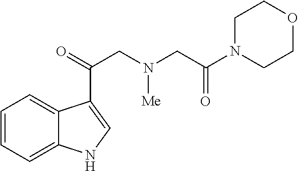 [SMILES: CN(CC(=O)c1c[nH]c2ccccc12)CC(=O)N1CCOCC1] |
| 2-(methyl(2-(2-phenyl-1H-indol-3- yl)ethyl)amino)-1-morpholinoethan-1-one | 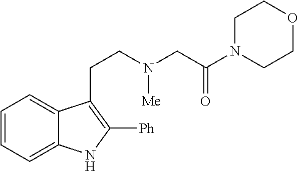 [SMILES: CN(CCc1c(-c2ccccc2)[nH]c2ccccc12)CC(=O)N1CCOCC1] |
| 1-(5-methoxy-2-phenyl-1H-indol-3-yl)-2- (methyl(2-morpholino-2-oxoethyl)amino)ethan- 1-one | 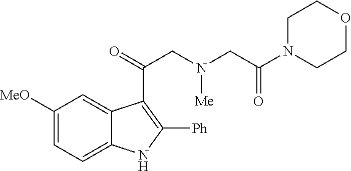 [SMILES: COc1ccc2[nH]c(-c3ccccc3)c(C(=O)CN(C)CC(=O)N3CCOCC3)c2c1] |
| 1-(2-(furan-2-yl)-1H-indol-3-yl)-2-(methyl(2- morpholino-2-oxoethyl)amino)ethan-1-one | 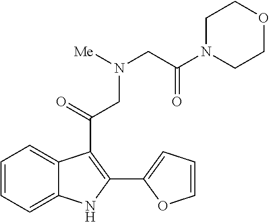 [SMILES: CN(CC(=O)c1c(-c2ccco2)[nH]c2ccccc12)CC(=O)N1CCOCC1] |
| N,N-dimethyl-2-(3-(2-(methyl(2-morpholino-2- oxoethyl)-amino)acetyl)-2-phenyl-1H-indol-5- yl)acetamide | 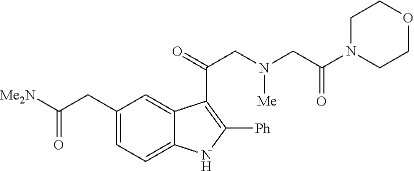 [SMILES: CN(CC(=O)c1c(-c2ccccc2)[nH]c2ccc(CC(=O)N(C)C)cc12)CC(=O)N1CCOCC1] |
| 1-(2-(hydroxymethyl)-1H-indol-3-yl)-2- (methyl(2-morpholino-2-oxoethyl)amino)ethan- 1-one | 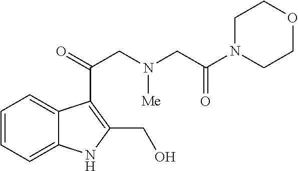 [SMILES: CN(CC(=O)c1c(CO)[nH]c2ccccc12)CC(=O)N1CCOCC1] |
| 2-((2-hydroxy-2-(2-phenyl-1H-indol-3- yl)ethyl)(methyl)amino)-1-morpholinoethan-1- one | 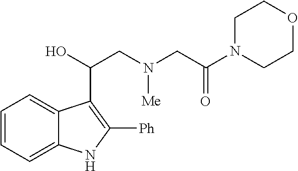 [SMILES: CN(CC(=O)N1CCOCC1)CC(O)c1c(-c2ccccc2)[nH]c2ccccc12] |
| N-methyl-N-(2-morpholino-2-oxoethyl)-2- phenyl-1H-indole-3-carboxamide | 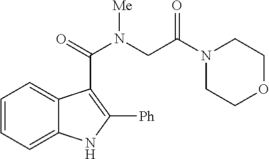 [SMILES: CN(CC(=O)N1CCOCC1)C(=O)c1c(-c2ccccc2)[nH]c2ccccc12] |
| 1-morpholino-2-((2-oxo-2-(2-phenyl-1H-indol- 3-yl)ethyl)amino)ethan-1-one | 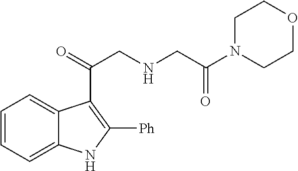 [SMILES: O=C(CNCC(=O)N1CCOCC1)c1c(-c2ccccc2)[nH]c2ccccc12] |
| 2-(methyl(2-oxo-2-(2-phenyl-1H-indol-3- yl)ethyl)amino)-1-(pyrrolidin-1-yl)ethan-1-one | 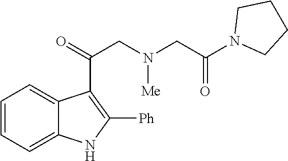 [SMILES: CN(CC(=O)c1c(-c2ccccc2)[nH]c2ccccc12)CC(=O)N1CCCC1] |
| N,N-diethyl-2-(methyl(2-oxo-2-(2-phenyl-1H- indol-3-yl)ethyl)amino)acetamide | 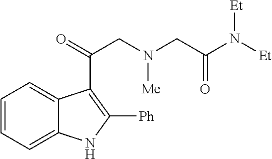 [SMILES: CCN(CC)C(=O)CN(C)CC(=O)c1c(-c2ccccc2)[nH]c2ccccc12] |
| 2-oxo-2-(2-phenyl-1H-indol-3-yl)ethyl-2-((2- hydroxyethyl)amino)benzoate | 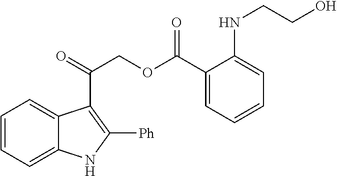 [SMILES: O=C(OCC(=O)c1c(-c2ccccc2)[nH]c2ccccc12)c1ccccc1NCCO] |
| 2-oxo-2-(2-phenyl-1H-indol-3-yl)ethyl 3- aminopyrazine-2-carboxylate | 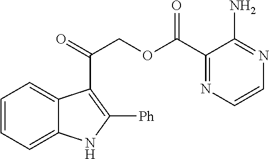 [SMILES: Nc1nccnc1C(=O)OCC(=O)c1c(-c2ccccc2)[nH]c2ccccc12] |

Representative GPRC6a antagonists which are compounds of formula (I) include, but are not limited to:
2-(methyl(2-morpholino-2-oxoethyl)amino)-1-(2-phenyl-1H-indol-3-yl)ethan-1-one;
1-(2-(4-methoxyphenyl)-1H-indol-3-yl)-2-(methyl(2-morpholino-2-oxoethyl)amino)ethan-1-one:
2-(methyl(2-morpholinoethyl)amino)-1-(2-phenyl-1H-indol-3-yl)ethan-1-one;
2-(3-(morpholine-4-carbonyl)piperidin-1-yl)-1-(2-phenyl-1H-indol-3-yl)ethan-1-one; and
1-(2-(4-fluorophenyl)-1H-indol-3-yl)-2-(methyl(2-morpholino-2-oxoethyl)amino)ethan-1-one,
   or a pharmaceutically acceptable salt thereof.

Compound names are assigned by using Struct=Name naming algorithm as part of CHEMDRAW® ULTRA v. 12.0.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Fumiss, Hannaford. Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quatemized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quatemary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. Administration

A composition may comprise the GPRC6a antagonist. The GPRC6a antagonists as detailed above can be formulated into a composition in accordance with standard techniques well known to those skilled in the pharmaceutical art. The composition may be prepared for administration to a subject. Such compositions comprising a GPRC6a antagonist can be administered in dosages and by techniques well known to those skilled in the medical arts taking into consideration such factors as the age, sex, weight, and condition of the particular subject, and the route of administration.

The GPRC6a antagonist can be administered prophylactically or therapeutically. In prophylactic administration, the GPRC6a antagonist can be administered in an amount sufficient to induce a response. In therapeutic applications, the GPRC6a antagonists are administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The GPRC6a antagonist can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The GPRC6a antagonist can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The GPRC6a antagonists can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the conjugate is administered intravenously, intraarterially, or intraperitoneally to the subject.

The GPRC6a antagonist can be a liquid preparation such as a suspension, syrup, or elixir. The conjugate can be incorporated into liposomes, microspheres, or other polymer matrices (such as by a method described in Felgner et al., U.S. Pat. No. 5,703,055: Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

### 4. METHODS

a. Methods of Treating a Condition in a Subject

The present invention is directed to methods of treating a condition in a subject in need thereof. The methods may include administering to the subject an effective amount of a GPRC6a antagonist. In some embodiments, the condition is selected from proteinopathy, Alzheimer's disease, tauopathy, Parkinson's disease, synucleinopathy, prion disease, amyloidosis, TDP-43, and neurodegenerative disease.

b. Methods of Inhibiting a GPRC6a in a Subject

The present invention is directed to methods of inhibiting a GPRC6a in a subject in need thereof. The methods may include administering to the subject an effective amount of a GPRC6a antagonist.

### 5. EXAMPLES

### Example 1

### Preliminary Findings

Our group recently uncovered a unique pathway between arginine metabolism, polyamine biology, and tau fate. L-arginine metabolism governs several systems including nitric oxide (NO) and polyamines (PAs). Arginase (Arg) or nitric oxide synthases (NOSes) metabolize L-arginine to generate omithine and subsequent PAs, or nitric oxide, respectively. PAs remain essential for growth; they interact with a variety of macromolecules, both electrostatically and covalently promoting different cellular effects. We find that physiological concentrations of PAs inhibit oligomerization/aggregation of tau in several models and demonstrate that arginine metabolism significantly alters the tau phenotype (J. B. Hunt, Jr., et al. J. Neurosci. 2015, 35, 14842). A recent report revealed a potential rare arginase-2 allele with increased risk of developing AD, while the enzyme ornithine transcarbamylase (OTC) could also be a minor genetic determinant of AD (F. Hansmannel, et al. J. Alzheimers Dis. 2010, 21, 1013). Several reports have previously shown altered PAs and arginine metabolism in CSF, plasma, or brain tissue in patients with mild cognitive impairment and AD. Untargeted blood-based metabolic profiling revealed that L-arginine and PA metabolism was disrupted between control patients, patients with mild cognitive impairment (MCI), and AD converters, which could be predicted up to two years for converters (S. F. Graham, et al. PLoS One 2015, 10, e0119452). These reports signify a relationship between arginine metabolism and AD. We find that arginase 1 (Arg1) overexpression in cell culture and animal models of tauopathy reduce many aspects of the tau phenotype and find similar outcomes in cell lines overexpressing tau with parallel Arg1 manipulations (J. B. Hunt, Jr., et al. J. Neurosci. 2015, 35, 14842). The following components of the tau phenotype decrease in response to Arg1 overexpression: reduced phospho-tau by neurohistological measures, reduced tangle pathology, reduced atrophy, reduced phospho-tau species and nitrated tau by neurochemical measures, reduced high molecular weight taui oligomers, reduced markers of inflammation, reduced inhibitors of autophagy, and reduced protein kinase activation. One mechanism for this may include L-arginine depletion and induction of autophagy through amino acid sensing.

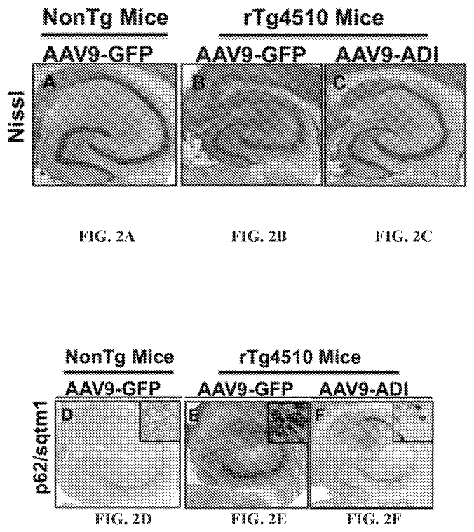

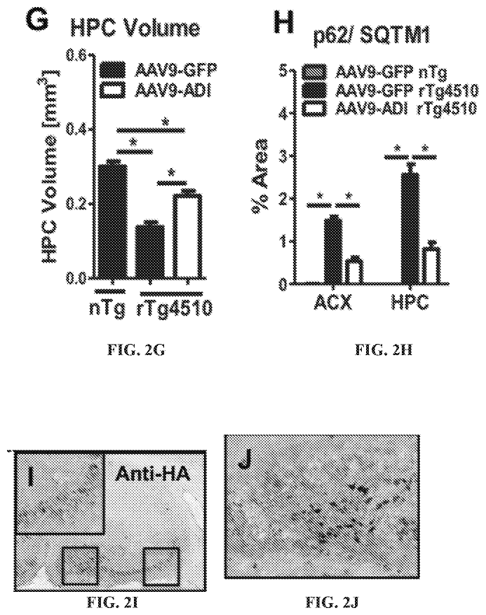

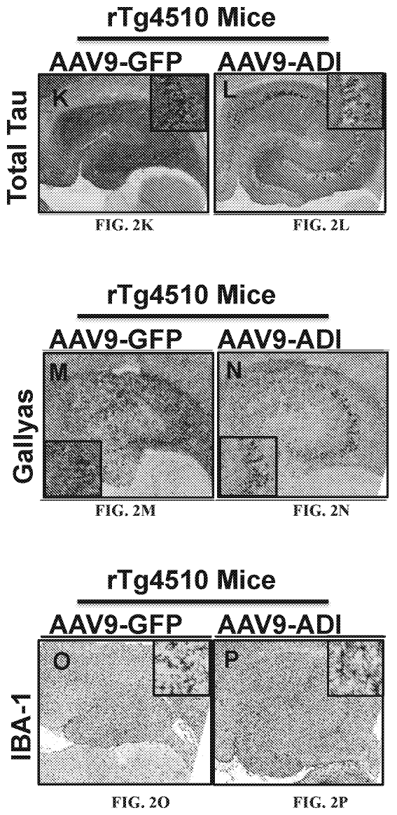

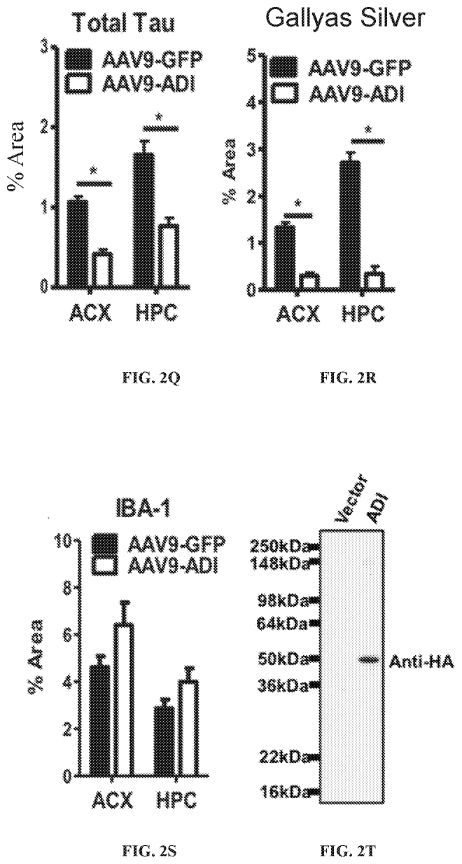

Although we also found that PAs decrease tau aggregation, the effects of increased PA production were not separated from the depletion of L-arginine through Arg1 overexpression. We successfully cloned, mammalianized (through codon usage), and expressed in the CNS (via recombinant adenoassociated virus) a bacterial enzyme known as arginine deiminase (ADI) to deplete L-arginine without increasing either nitric oxide or PAs, but instead produces citrulline. The ADI clone signifies a novel tool to study the effects of L-arginine metabolism. Essentially, we created a new pathway in the mammalian brain. We tested our synthetic gene ADI in rTg4510 tau transgenic mice. A control virus (GFP) rAAV9-GFP and rAAV9-ADI (HA-tagged) was injected in the hippocampus and anterior cortex of aged (12-month-old) rTg4510 mice (an available cohort in our colony). After two months of treatment we shockingly found that ADI (n=8) (FIG. 2I, FIG. 2J, FIG. 2T) dramatically reduced hippocampal atrophy (FIG. 2A, FIG. 2B, FIG. 2C, FIG. 2G) and p62 (FIG. 2D. FIG. 2E, FIG. 2F, FIG. 2H) compared to GFP, suggesting induction of autophagy. We also found a reduction in total tau (FIG. 2K, FIG. 2L. FIG. 2Q), tauSer396, AT8 (not shown), tangles (Gallyas silver) (FIG. 2M, FIG. 2N, FIG. 2R), and no change in IBA-1 (FIG. 2O, FIG. 2P, FIG. 2S) (although slightly elevated) compared to GFP (n=7) likely due to the foreign gene. These data suggest that arginine metabolism may play a vital role in tau clearance through amino acid sensing.

This led us to search for receptors that regulate putative arginine signaling. Interestingly. GPRC6a is a family C G-protein coupled receptor recently discovered, cloned, deorphanized (P. Wellendorph, et al. Molecular pharmacology 2005, 67, 589) and shown to bind L-α amino acids, particularly basic amino acids including L-arginine and omithine. Ligands (L-arginine, ornithine etc.) may tonically signal through GPRC6a amino acid “sufficiency” and slow the rate of autophagy. However, as L-arginine/omithine levels decline or “signaling” decreases, induction of autophagy occurs. ADI's efficacy may derive from depletion of both L-arginine and omithine, thereby promoting tau clearance even in 12-month old rTg4510 mice. The lysosomal amino acid transporter SLC38A9 also signals L-arginine sufficiency to mTORC1, supporting the idea of L-arginine as a critical molecule for regulating autophagy. Decreased signaling of GPRC6a or allosteric antagonism to GPRC6a may activate autophagy and tau clearance. GPRC6a may remain tonically activated and senses the amino acids abundance, but perhaps is more sensitive to L-arginine and omithine. Allosteric antagonism (or genetic targeting) of GPRC6a essentially reduces the efficacy and functionality of the receptor, thereby nullifying endogenous ligands ineffective and signaling “amino acid deficiency” inducing autophagy. We will exploit this potential mechanism to clear tau deposits. This would comprise the first GPCR linked to autophagy through amino acid sensing. We will test if modulation of GPRC6a impacts the tau phenotype by three approaches: genetic knockout of GPRC6a, targeted knock down of GPRC6a by gene therapy, and pharmacological antagonism to GPRC6a with novel drugs.

### Example 2

### Decreased Signaling of GPRC6a Increased Tau and Alpha Synuclein Clearance

Utilizing novel allosteric antagonists to GPRC6a, we found clearance of tau in primary cortico-hippocampal neurons, stably overexpressing tau cell lines, alpha synuclein cell lines, and P301S tau transgenic mice. To the extent that this intracellular pathway associates with autophagy and protein clearance through amino sensing, GPRC6a signaling may impact proteostasis and neurodegenerative diseases. We have identified 3 lead compounds that harbor the 2-phenylindole privileged structure scaffold in which reduce tau or alpha synuclein expression in stably overexpressing cells but have also generated more than 70 derivatives in this structural class of compounds as allosteric antagonist GPRC6a ligands.

### Example 3

### To Determine if GPRC6a Knockout Mice Show Reduced Accumulation of Tau Pathology

We will test whether GPRC6a deletion promotes tau clearance mediated by adeno associated viral (rAAV) tau overexpression compared to wild-type littermates. We will measure aspects of the tau phenotype including behavioral impairment in GPRC6a knockout mice and wild type littermates. GPRC6a knockout mice will show greater activation of autophagy when exposed to tau.

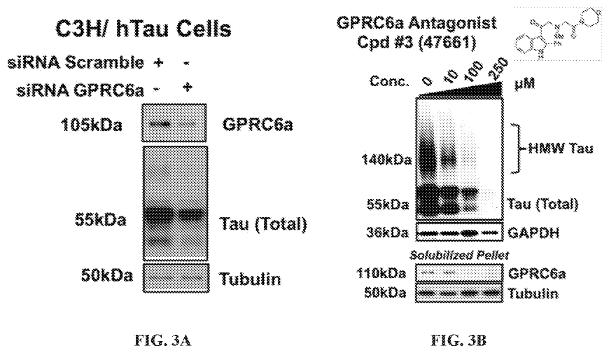

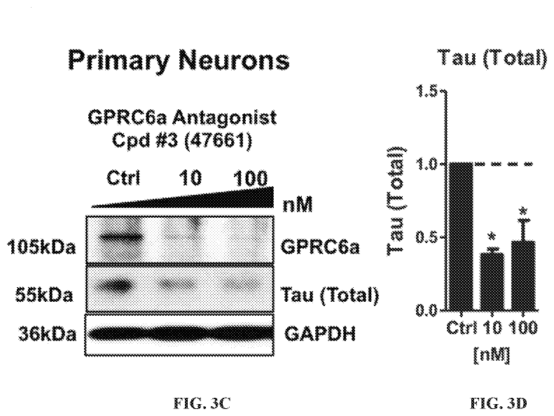

Our previous work showed that L-arginine increased mammalian target of rapamycin (mTOR) and tau levels in cells but Arg1 overexpression decreased both mTOR and tau. Conditional deletion of Arg1l in myeloid/microglia cells (LysMCre Arg1), accumulates more tau than Arg1 sufficient mice using tau C-terminally truncated at D421 (rAAV-cTau-D421), which is preferentially cleared through autophagy. This suggests that decreased Arg1 (or arginine accumulation) promotes tau pathology. We also showed higher Larginine levels in rTg4510 mice and that tau (D421) reduced Arg1 mRNA levels suggesting that L-arginine impacts tau expression but also that tau impairs arginine metabolism (J. B. Hunt, Jr., et al. J. Neurosci. 2015, 35, 14842). We will test rAAV-cTau-D421 overexpression in GPRC6a knockout mice. rAAV-cTau-D421 showed increased tau pathology (total tau, phospho Ser199/202, others not shown) and cognitive impairment (decreased working [Ymaze], spatial working memory [radial arm water maze], and fear associated memory [inhibitory avoidance]) compared to control virus or empty capsid (FIG. 3)

We will use two genotypes of mice aged 6 months (mo): Group 1 (GPRC6a+/+) wild-type littermates; Group 2 (GPRC6a−/−) ko mice. Each group will receive bilateral injection into the hippocampus (HPC) and anterior cortex (ACX) of either rAAV-GFP or rAAV-cTau (D421) for a viral duration of 4 months. We will use a naïve set of wild-type littermates and GPRC6a ko mice to compare the effect of viral overexpression. We will use a sample size of 12 mice per group and balance both genders and litters with respect to experimental group assignment. We will use convection-enhanced delivery (CED) of tau to maximize the extent of vector diffusion (described in N. Carty, et al. J. Neurosci. Methods 2010, 194, 144). At 10 months of age mice will receive a battery of behavioral task including (in series of least stressful to more stressful): open field, rotarod, Y-maze, elevated plus maze, radial arm water maze (RAWM), and inhibitory avoidance. We will confirm levels of transduced gene expression by western or ELISA methods. We will process tissue as detailed in J. B. Hunt, Jr., et al. J. Neurosci. 2015, 35, 14842. We will use histological and neurochemical dependent measures to assess the tau phenotype including NeuN and Nissl staining for neuronal counts and brain volume by stereology, synaptophysin, total tau, phospho-tau deposition (AT8, pSer199/202, pSer356), tangle pathology, high molecular weight tau (T22 oligomers), autophagy markers LC3B, mTOR, p62, beclin, microglia activation (IBA-1, CD45, CD68, TSPO), arginine, ornithine, citrulline agmatine levels. TSPO is a marker of inflammation in our panel, which corresponds to the only marker used to measure inflammation in human brain. PK-11195 binds TSPO, and all new PET agents in development are also directed at this target. It will be important to learn how this marker changes with phenotype and treatment to inform studies in humans regarding target engagement.

GPRC6a knockout mice will show greater activation of autophagy compared to wild-type littermates when exposed to tau. Further, there will be less tau accumulation in GPRC6a knockout mice compared to wild-type littermates. This would indicate that decreased GPRC6a signaling promotes tau clearance and validate the receptor as a potential target for tauopathies and AD. Should we find the opposite, that is GPRC6a ko mice accumulate more tau, it might suggest an alternative or compensatory pathway of L-arginine signaling and autophagy (i.e. SLC38A9). We would then measure the expression/localization of the SLC38A9 lysosomal transporter in response to tau.

For most experiments, group performance will be analyzed using a 2×2 Factorial ANOVA (Genotype x Treatment) followed by pair-wise comparisons for genotype-specific effects of treatment. Independent student's t-tests will be used to examine specific pairings not covered in the 2×2 Factorial ANOVA. We will analyze histopathology data by ANOVA followed by posthoc means comparisons using the Tukey's multiple comparison (SPSS/GraphPad Prism 5.0).

### Example 4

### To Determine if rAAV-Mediated shRNA Knockdown of GPRC6a Reduces the Tau Phenotype

We will test the hypothesis that viral mediated shRNA knockdown of GPRC6a induces autophagy and mitigates tau neuropathology in rTg4510 transgenic mice. We will measure aspects of the tau phenotype in addition to behavioral impairments in rTg4510 transgenic mice. Reduction in the GPRC6a will also exhibit greater activation of autophagy in tau transgenic mice.

rTg4510 tau mice comprise of the tau P301L mutation which expresses in the forebrain and hippocampus (K. Santacruz, et al. Science 2005, 309, 476). Mice showed age-dependent increases in ptau isoforms, including insoluble Gallyas positive filaments, increased glial activation, decreased synaptic density, impaired synaptic plasticity, memory loss and ultimately neuronal loss and regional atrophy (J. B. Hunt, Jr., et al. J. Neurosci. 2015, 35, 14842; K. Santacruz, et al. Science 2005, 309, 476; C. Dickey, et al. Am. J. Palhol. 2009, 174, 228). Accumulation of ptau occurs largely between 3 and 9 mo of age. Additionally, high-molecular weight tau multimers/oligomers and nitrated tau also accumulate in rTg4510 tau transgenic mice compared to non-transgenic littermates (J. B. Hunt, Jr., et al. J. Neurosci. 2015, 35, 14842). We demonstrated that rTg4510 tau mice show significant p62 accumulation in neurons suggesting impairment of autophagy (J. B. Hunt, Jr., et al. J. Neurosci. 2015, 35, 14842). In stably tau overexpressing HeLa cells (C3H/htau), siRNA to GPRC6a reduced receptor expression coupled with a reduction in tau (FIG. 4). We will repress GPRC6a expression in the CNS with rAAV-shRNA-GPRC6a to reduce receptor signaling.

We will inject rTg4510 mice and non-transgenic littermates at 4 months of age at four sites (both hippocampi and both anterior cortices) with AAV9 vectors designed to express (A) rAAV-shRNA-GPRC6a (with a GFP transduction monitor) (Origene™), and (B) rAAV-shRNA-scramble-GFP construct (control group). We will use an untreated group of rTg4510 mice and non-transgenic littermates to ascertain the impacts of these constructs on the normal phenotypes. Experimental group assignments, dependent measures and statistical analyses will be the same as in Example 1.

Autophagy will be activated in rTg4510 tau mice following shRNA repression of GPRC6a compared to the control construct. Again, this would confirm our central hypothesis and show GPRC6a as a viable target for tauopathies and AD. In contrast, Example 1 provides complete knockout of GPRC6a during a pathogenic form of tau, while this provides regional CNS knockdown of the receptor during tau pathology.

### Example 5

### To Identify if Selective GPRC6a Allosteric Antagonists Increase Tau Degradation and Clearance

We will examine whether novel and selective GPRC6a allosteric antagonists increase autophagy and tau clearance in vitro. We will test novel GPRC6a antagonists in an inducible tetOn tau-Dendra2 photoswitchable cell line to measure degradation kinetics of tau. We will also measure autophagy signaling by western blot analysis. Efficacy and signaling of GPRC6a will be decreased, thereby inducing autophagy in cells. Success in this application would provide a new receptor target that activates autophagy through amino acid sensing and may reduce pathology and mitigate the tau phenotype.

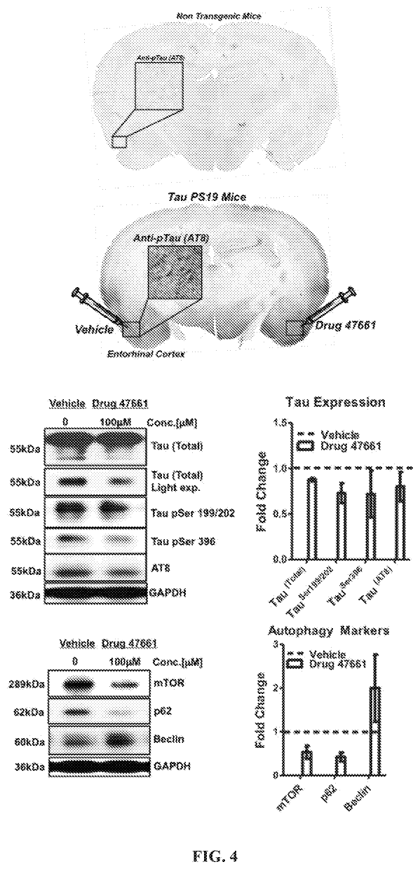

We used an allosteric antagonist to GPRC6a (compound 3) (D. E Gloriam, et al. Chem. Biol. 2011, 18, 1489) referred to as (Drug 47661), and observed significant clearance among monomeric, insoluble, and oligomeric tau in C3H/htau cells (micromolar efficacy) (FIG. 4). Drug 47661 also reduced endogenous tau in primary neurons (nanomolar efficacy), FIG. 4). Finally, a one-time bolus injection of Drug 47661 (78 ng) into the entorhinal cortex (early stage tau deposits) of 8 month-old PS 19 tau (P301S) transgenic mice modestly reduced tau expression after 72 hours (FIG. 4). We will test several newly generated, novel and more selective GPRC6a allosteric antagonists to determine the impact on tau clearance in vitro. Some of the compounds have 9-fold more selectivity for GPRC6a than our current compound in a FRET-based assay (H. Johansson, et al. J. Med. Chem. 2015, 58, 8938), and an excess of 50 derivatives to these current lead compounds will be analyzed. A tetracycline inducible “on” tau fusion-Dendra2 stable cell line will be used, which is a green-to-red irreversible photoswitchable fluorescent protein activated by UV-violet/blue light. We will induce tau expression with tetracycline (1 μg/mL) for 24 h, then photo convert tau dendra2-green with blue light (488 nm) for 10 minutes to tau-dendra2-red for monitoring kinetics of tau. FIG. 5 shows the tau-Dendra2 photoconversion from green to red after 10 min of blue light. We will treat cells with GPRC6a allosteric antagonists for 72 hours with continual red fluorescent output readings every 6 hours (553 nm ex and 573 nm em). This assay will measure tau degradation kinetics over time (time 0 point at which photo switch occurs from tau-green to tau-red) (FIG. 5). Agents that govern tau biology will alter the rate of tau degradation (tau-red). Newly synthesize tau (tau-green) will fluoresce green to be measure on a separated channel. We will also co-label cells with DAPI nuclear stain to account for cell loss and nuclear chromatin changes. We will use Biotek®@ Cytation™ 3 Cell Imaging Multi-Mode Reader located at Byrd Institute. We will use a 96 well semi-high throughput platform with GPRC6a antagonist concentrations ranging from (10 nM-100 μM). We will also perform cell toxicity assays (Cyto Tox-ONE LDH Membrane Integrity Assay, Promega). We will perform western blot analysis in C3H/htau cells from GPRC6a antagonists that reduce tau levels more than 30% at 3 μM or below and with no more than 5-10% toxicity. We will measure autophagy related makers to determine the mechanism for tau reduction.

Following GPRC6a antagonists, efficacy of GPRC6a will be decreased, signaling will be reduced, and therein induction of autophagy in cells will be reduced. One limitation to the Dendra2 assay is that following toxicity, tau-red is released into the media and can account for higher background levels of fluorescence. Again, we will exclude those data (concentrations) in which toxicity occurs. Cytation™ 3 provides images for each well to confirm output and correlate numeric data. This application will provide a new receptor target and class of drugs for AD and tauopathies.

### Example 6

### Additional Studies

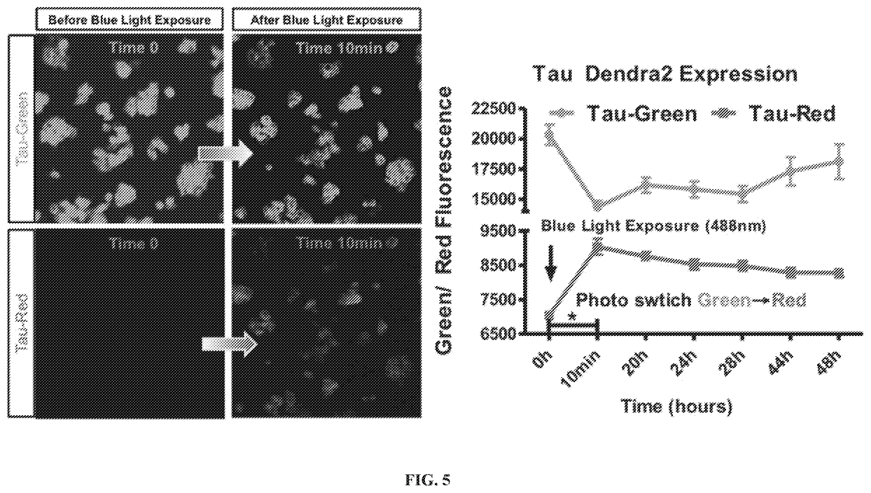

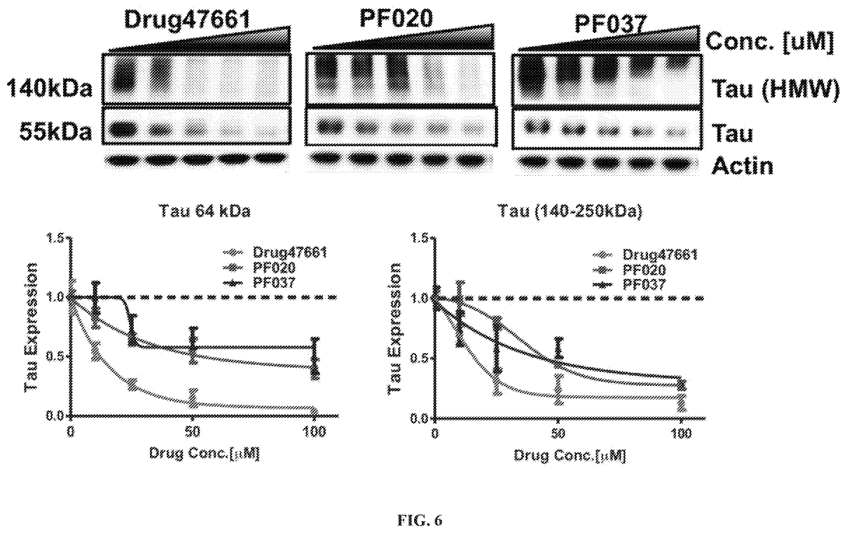

C3H/htau cells were treated for 72 hours with varying concentrations of GPRC6A antagonist Drug 47661, PF020, or PF037. The GPRC6A antagonists studied are shown in TABLE 2. Samples were run on a gel. As shown in FIG. 6, the cells demonstrated decreased tau expression upon treatment with GPRC6A antagonist, as compared to vehicle. All three GPRC6a allosteric antagonists tested decreased monomeric and high molecular weight (HMW) tau multimers in the C3H/htau cells.

TABLE 2
GPRC6A antagonists examined with C3H/htau cells.

| Name | Structure | MW (g/mol) | Amount (mg) |
|---|---|---|---|
| 47661 | 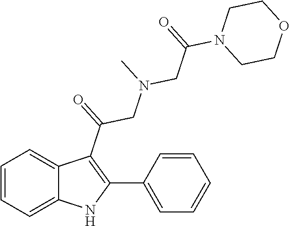 [SMILES: CN(CC(=O)c1c(-c2ccccc2)[nH]c2ccccc12)CC(=O)N1CCOCC1] | 391.19 | 2.43 |
| PF020 | 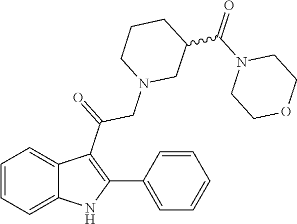 [SMILES: O=C(CN1CCCC(C(=O)N2CCOCC2)C1)c1c(-c2ccccc2)[nH]c2ccccc12] | 431.22 | 2.82 |
| PF037 | 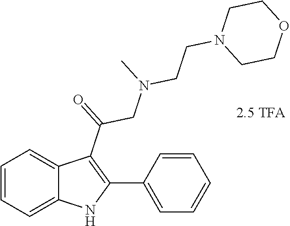 [SMILES: CN(CCN1CCOCC1)CC(=O)c1c(-c2ccccc2)[nH]c2ccccc12] | 672.54 | 1.34 |

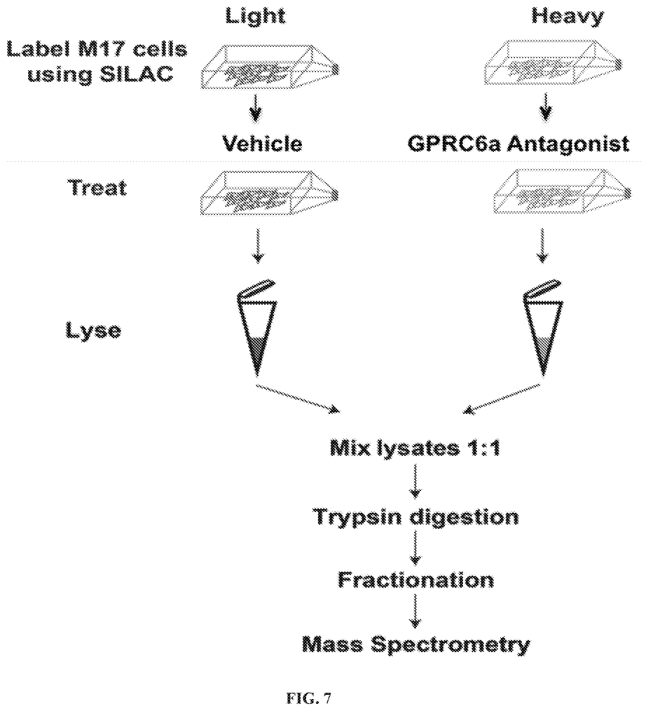

The effect of the GPRC6A antagonists on protein expression in cells will be analyzed using mass spectrometry. The overall procedure for SILAC based proteomics is shown in FIG. 7. M16 cells will be grown and passaged in medium containing heavy and light amino acids using the Pierce SILAC Protein Quantitation Kit (Thermo Fisher Scientific, Waltham, Mass.). CM16 cells will be then treated with GPRC6a antagonist, vehicle, GPRC6a siRNA, or scramble siRNA. Lysates will be mixed together, digested, fractioned, and analyzed by mass spectrometry. Data from the spectrometer will be processed using MaxQuant and Ingenuity Pathway Analysis (IPA).

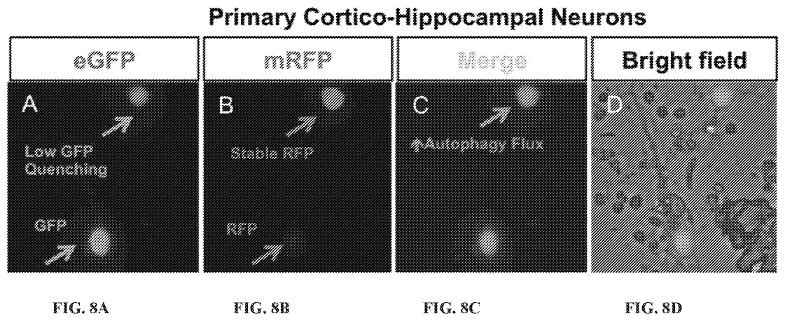

Autophagy was examined in primary mouse cortico-hippocampal neurons. Primary mouse cortico-hippocampal neurons were transfected with a LC3-mRFP-eGFP tandem plasmid construct for 48 hours (FIG. 8A-FIG. 8D). The LC3-RFP/GFP tandem construct exploits differences between GFP (which is fluorescently quenched in lysosomes) and RFP (stable RFP fluorescence in lysosomes). Cells with more autophagosomes would be labeled with yellow signal (i.e. RFP and GFP) and their maturation into autolysosomes would be labeled with a red/orange signal (i.e. RFP only, after quenching of GFP fluorescence in the lysosome), thereby being an indicator of autophagy activity. As shown in FIG. 8E-FIG. 8G, primary neurons demonstrated a uniform difference in high versus low GFP and RFP fluorescence, indicating different degrees of autophagy flux. FIG. 8E-FIG. 8G represent mouse HT22 cells (immortalized hippocampal cell line) transfected with the LC3-RFP/GFP tandem construct, and show a more heterogenic population, which exhibited various degrees of GFP/RFP expression (but overall yellow) in different compartments and organelles also indicating different degrees of autophagy flux.

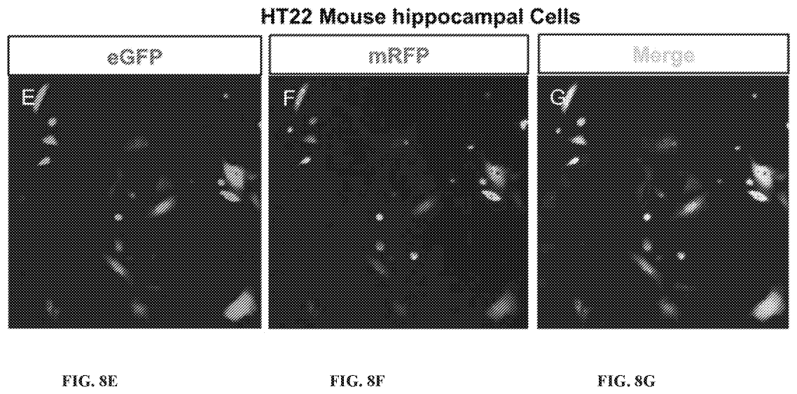

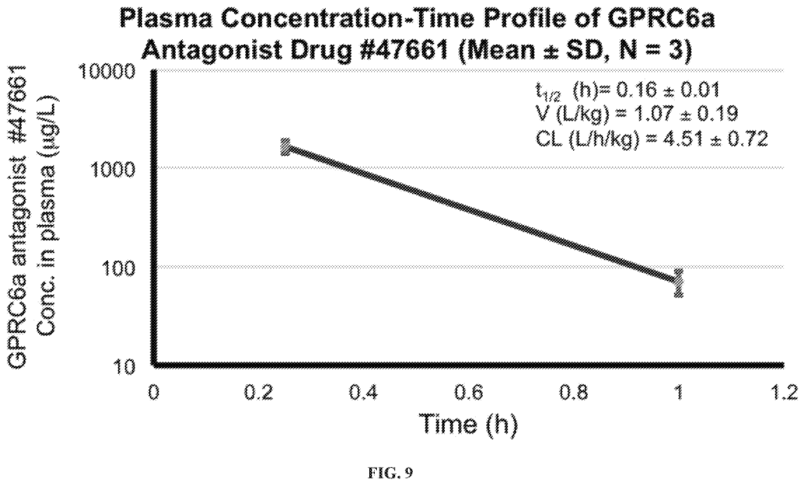

The half-life of GPRC6A antagonist Drug 47661 was examined. Naïve mice were injected with 5 mg/kg of Drug 47661 by IV. Serial submandibular bleeds were taken 15 min and 60 min post IV injection. The half-life (0.16 h), volume of distribution (1.07 L/kg), and clearance rate (4.5 L/h/kg) were determined for Drug 47661. (N=3). Shown in FIG. 9 is a graph showing the relative half-life of Drug 47661.

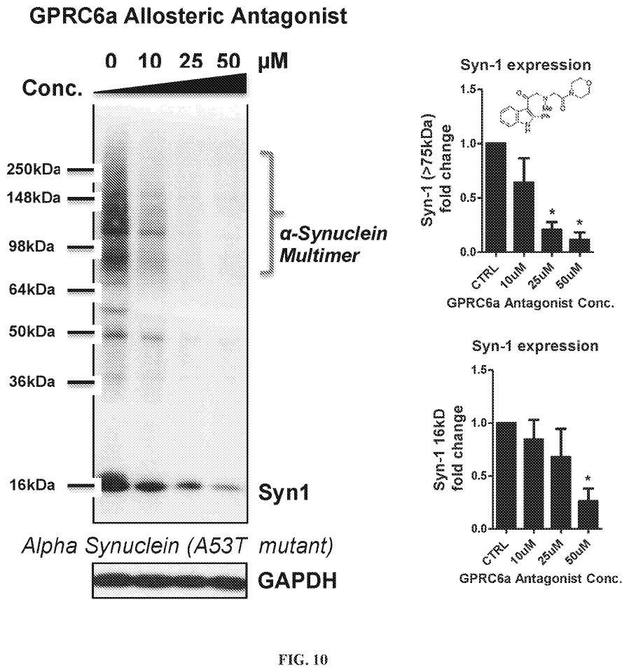

BE(2) M17 cells were stably transfected with wild-type alpha synuclein (16 kDa) and treated with GPRC6a antagonist drug 47661 for 72 hours. Samples were run on a gel. As shown in FIG. 10, Drug 47661 decreased monomeric (16 kDa) and oligomeric (>75 kDa) alpha synuclein expression, suggesting an increased clearance or degradation of the protein relative to GAPDH as a protein loading control. Statistical analysis was performed using One-way Anova with Fisher's LSD multiple comparison test as post hoc.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of treating a condition in a subject, the method comprising administering to the subject in need thereof an effective amount of a GPRC6a antagonist.

Clause 2. The method of clause 1, wherein the condition comprises a proteinopathy.

Clause 3. The method of clause 2, wherein the proteinopathy comprises a neurodegenerative disease.

Clause 4. The method of clause 2, wherein the proteinopathy comprises a tauopathy, synucleinopathy, prion disease, or amyloidosis.

Clause 5. The method of clause 4, wherein the tauopathy is selected from primary age-related tauopathy (PART)/Neurofibrillary tangle-predominant senile dementia, chronic traumatic encephalopathy including dementia pugilistica, progressive supranuclear palsy, Pick's Disease, corticobasal degeneration, some forms of frontotemporal lobar degeneration, frontotemporal dementia and parkinsonism linked to chromosome 17, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis. Hallervorden-Spatz disease, lipofuscinosis, Huntington's Disease, and Alzheimer's Disease (AD).

Clause 6. The method of clause 4, wherein the synucleinopathy is selected from Parkinson's Disease, dementia with Lewy bodies, neuroaxonal dystrophies, and multiple system atrophy.

Clause 7. A method of inhibiting a GPRC6a in a subject in need thereof, the method comprising administering to the subject a GPRC6a antagonist.

Clause 8. The method of any one of clauses 1-7, wherein the GPRC6a antagonist is a compound of formula (I), or a pharmaceutically acceptable salt thereof.

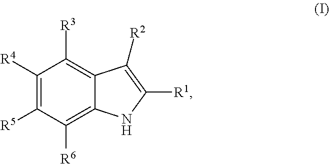

wherein R¹ is hydrogen, alkyl, aryl, cycloalkyl, heteroaryl, or heterocycle, wherein the alkyl, aryl, cycloalkyl, heteroaryl, and heterocycle are each optionally substituted with one or more substituents selected from the group consisting of —OH, alkoxy, —NR^{1a}R^{1b}, halogen, nitro, —C(O)-alkyl, —C(O)—O-alkyl, —C(O)—NR^{1a}R^{1b}; R² is —X—(CR^{x}R^{y})ₘ₁—Y—(CR^{x}R^{y})ₘ₂—Z; R³, R⁴, R⁵, R⁶ are independently hydrogen, alkyl, halogen, nitro, alkoxy, or alkyl substituted with —CO—R^{3a}, —CO—OR^{3a}, or —CO—NR^{3a}R^{3b}, wherein X is —CH₂—, —CH(OH)—, or —CO—; Y is —O— or —NR^{2a}—; Z is hydrogen. -G, or —CO-G, wherein G is an optionally substituted aryl, optionally substituted cycloalkyl, optionally substituted heteroaryl, or optionally substituted heterocycle; m1 is 0-10; m2 is 0-10; R^{1a}, R^{1b}, R^{3a}, and R^{3b} at each occurrence are independently hydrogen or alkyl; and R^{2a}, R^{x} and R^{y} at each occurrence are independently hydrogen or alkyl, or R^{2a} and one R^{x}, together with the N to which R^{2a} is attached and the C to which R^{x} is attached, form a 5-membered or 6-membered heterocycle.

Clause 9. The method of clause 8, wherein the GPRC6a antagonist is a compound of formula (I-a), or a pharmaceutically acceptable salt thereof,

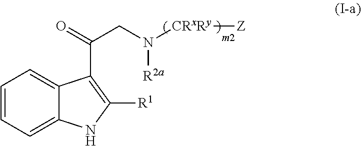

wherein R¹ is optionally substituted aryl, optionally substituted cycloalkyl, optionally substituted heteroaryl, or optionally substituted heterocycle; and Z is -G or —CO-G.

Clause 10. The method of clause 9, wherein R¹ is optionally substituted aryl.

Clause 11. The method of clause 9, wherein R^{2a} is alkyl.

Clause 12. The method of clause 9, wherein R¹ is phenyl or phenyl substituted with one or more alkoxy or halogen; R^{2a} is alkyl, or R^{2a} and one R^{x}, together with the N to which R^{2a} is attached and the C to which R^{x} is attached, form a 5-membered or 6-membered heterocycle.

Clause 13. The method of clause 9, wherein R¹ is phenyl, and R^{2a} is alkyl.

Clause 14. The method of clause 9, wherein R¹ is phenyl, and R^{2a} and one R^{x}, together with the N to which R^{2a} is attached and the C to which R^{x} is attached, form a 5-membered or 6-membered heterocycle.

Clause 15. The method of clause 9, wherein R¹ is phenyl substituted with one or more alkoxy or halogen, and R^{2a} is alkyl.

Clause 16. The method of any one of clauses 9-15, wherein m2 is 1, 2, 3, or 4.

Clause 17. The method of clause 9, wherein the GPRC6a antagonist is a compound of formula (I-a1), (I-a2), or (I-a3), or a pharmaceutically acceptable salt thereof,

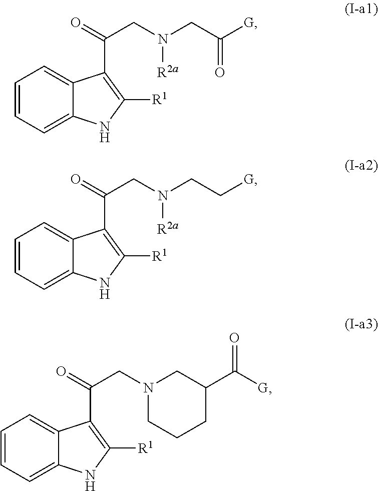

Clause 18. The method of clause 17, wherein R¹ is phenyl or phenyl substituted with one or more alkoxy or halogen.

Clause 19. The method of clause 8, wherein the GPRC6a antagonist is a compound of formula (I-b), or a pharmaceutically acceptable salt thereof,

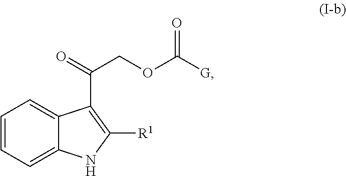

Clause 20. The method of any one of clauses 8-19, wherein G is

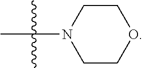 [SMILES: *N1CCOCC1]

Clause 21. The method of clause 8, wherein the compound is selected from the group consisting of 2-(methyl(2-morpholino-2-oxoethyl)amino)-1-(2-phenyl-1H-indol-3-yl)ethan-1-one; 1-(2-(4-methoxyphenyl)-1H-indol-3-yl)-2-(methyl(2-morpholino-2-oxoethyl)amino)ethan-1-one; 2-(methyl(2-morpholinoethyl)amino)-1-(2-phenyl-1H-indol-3-yl)ethan-1-one; 2-(3-(morpholine-4-carbonyl)piperidin-1-yl)-1-(2-phenyl-1H-indol-3-yl)ethan-1-one; and 1-(2-(4-fluorophenyl)-1H-indol-3-yl)-2-(methyl(2-morpholino-2-oxoethyl)amino)ethan-1-one, or a pharmaceutically acceptable salt thereof.

Clause 22. The method of any one of the preceding clauses, wherein the GPRC6a antagonist increases clearance of tau.

Clause 23. The method of any one of the preceding clauses, wherein the GPRC6a antagonist reduces tau and/or alpha synuclein expression.

Clause 24. The method of any one of the preceding clauses, wherein the GPRC6a antagonist reduces or clears multiple forms of tau and/or alpha synuclein.

Clause 25. The method of clause 24, wherein the multiple forms are selected from insoluble, monomeric, and high molecular weight multimers.

Clause 26. The method of any one of the preceding clauses, wherein the GPRC6a antagonist increases or promotes the clearance of pathogenic aggregation-prone proteins.

1. A method of treating a condition comprising a proteinopathy in a subject, the method comprising administering to the subject in need thereof an effective amount of a GPRC6a antagonist.

2. (canceled)

3. The method of claim 1, wherein the proteinopathy comprises a neurodegenerative disease.

4. The method of claim 1, wherein the proteinopathy is selected from a tauopathy, synucleinopathy, prion disease, amyloidosis, or a combination thereof.

5. The method of claim 4, wherein the tauopathy is selected from primary age-related tauopathy (PART)/Neurofibrillary tangle-predominant senile dementia, chronic traumatic encephalopathy including dementia pugilistica, progressive supranuclear palsy, Pick's Disease, corticobasal degeneration, some forms of frontotemporal lobar degeneration, frontotemporal dementia and parkinsonism linked to chromosome 17, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, Huntington's Disease, and Alzheimer's Disease (AD).

6. The method of claim 4, wherein the synucleinopathy is selected from Parkinson's Disease, dementia with Lewy bodies, neuroaxonal dystrophies, and multiple system atrophy.

7. A method of inhibiting a GPRC6a in a subject in need thereof, the method comprising administering to the subject a GPRC6a antagonist.

8. The method of claim 1, wherein the GPRC6a antagonist is a compound of formula (I), or a pharmaceutically acceptable salt thereof, 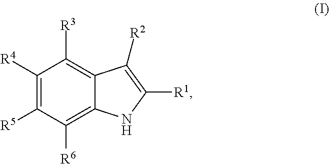
wherein
R¹ is hydrogen, alkyl, aryl, cycloalkyl, heteroaryl, or heterocycle, wherein the alkyl, aryl, cycloalkyl, heteroaryl, and heterocycle are each optionally substituted with one or more substituents selected from the group consisting of —OH, alkoxy, —NR^{1a}R^{1b}, halogen, nitro, —C(O)-alkyl, —C(O)—O-alkyl, —C(O)—NR^{1a}R^{1b};
R² is —X—(CR^{x}R^{y})ₘ₁—Y—(CR^{x}R^{y})ₘ₂—Z;
R³, R⁴, R⁵, R⁶ are independently hydrogen, alkyl, halogen, nitro, alkoxy, or alkyl substituted with —CO—R³⁸, —CO—OR^{3a}, or —CO—NR^{3a}R^{3b},
wherein
X is —CH₂—, —CH(OH)—, or —CO—;
Y is —O— or —NR^{2a}—;
Z is hydrogen, -G, or —CO-G, wherein G is an optionally substituted aryl, optionally substituted cycloalkyl, optionally substituted heteroaryl, or optionally substituted heterocycle;
m1 is 0-10;
m2 is 0-10;
R^{1a}, R^{1b}, R^{3a}, and R^{3b} at each occurrence are independently hydrogen or alkyl; and
R^{2a}, R^{x}, and R^{y} at each occurrence are independently hydrogen or alkyl, or R^{2a} and one R^{x}, together with the N to which R^{2a} is attached and the C to which R^{x} is attached, form a 5-membered or 6-membered heterocycle.

9. The method of claim 8, wherein the GPRC6a antagonist is a compound of formula (I-a), or a pharmaceutically acceptable salt thereof, 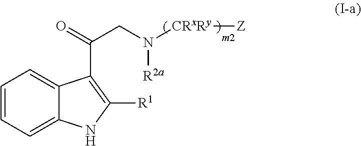
wherein
R¹ is optionally substituted aryl, optionally substituted cycloalkyl, optionally substituted heteroaryl, or optionally substituted heterocycle; and
Z is -G or —CO-G;

10. The method of claim 9, wherein R¹ is optionally substituted aryl.

11. The method of claim 9, wherein R^{2a} is alkyl.

12. The method of claim 9, wherein
R¹ is phenyl or phenyl substituted with one or more alkoxy or halogen; and
R^{2a} is alkyl, or R² and one R^{x}, together with the N to which R^{2a} is attached and the C to which R^{x} is attached, form a 5-membered or 6-membered heterocycle.

13. (canceled)

14. The method of claim 9, wherein
R¹ is phenyl; and
R^{2a} and one R^{x}, together with the N to which R^{2a} is attached and the C to which R^{x} is attached, form a 5-membered or 6-membered heterocycle.

15. The method of claim 9, wherein
R¹ is phenyl substituted with one or more alkoxy or halogen; and
R^{2a} is alkyl.

16. The method of claim 9, wherein m2 is 1, 2, 3, or 4.

17. The method of claim 9, wherein the GPRC6a antagonist is a compound of formula (I-a1), (I-a2), or (I-a3), or a pharmaceutically acceptable salt thereof,

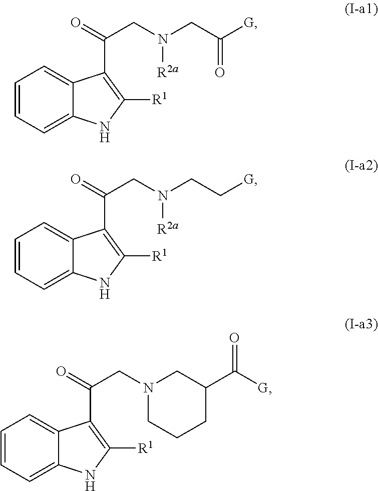

18. The method of claim 17, wherein R¹ is phenyl or phenyl substituted with one or more alkoxy or halogen.

19. The method of claim 8, wherein the GPRC6a antagonist is a compound of formula (I-b), or a pharmaceutically acceptable salt thereof,

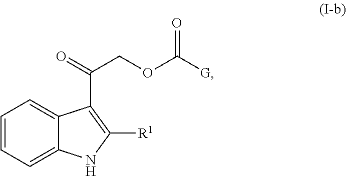

20. The method of claim 8, wherein G is

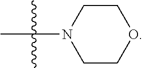 [SMILES: *N1CCOCC1]

21. The method of claim 8, wherein the compound is selected from the group consisting of
2-(methyl(2-morpholino-2-oxoethyl)amino)-1-(2-phenyl-1H-indol-3-yl)ethan-1-one;
1-(2-(4-methoxyphenyl)-1H-indol-3-yl)-2-(methyl(2-morpholino-2-oxoethyl)amino)ethan-1-one;
2-(methyl(2-morpholinoethyl)amino)-1-(2-phenyl-1H-indol-3-yl)ethan-1-one;
2-(3-(morpholine-4-carbonyl)piperidin-1-yl)-1-(2-phenyl-1H-indol-3-yl)ethan-1-one; and
1-(2-(4-fluorophenyl)-1H-indol-3-yl)-2-(methyl(2-morpholino-2-oxoethyl)amino)ethan-1-one,
or a pharmaceutically acceptable salt thereof.

22. The method of claim 1, wherein the GPRC6a antagonist increases clearance of tau, reduces tau and/or alpha synuclein expression, reduces or clears multiple forms of tau and/or alpha synuclein, increases or promotes the clearance of pathogenic aggregation-prone proteins, or a combination thereof.

23. (canceled)

24. (canceled)

25. The method of claim 22, wherein the multiple forms are selected from insoluble, monomeric, and high molecular weight multimers.

26. (canceled)
